(19) United States
(12) Patent Application Publication
(10) Pub. No.: US 2016/0132726 A1
(43) Pub. Date: May 12, 2016

(54) SYSTEM AND METHOD FOR ANALYSIS OF EYE MOVEMENTS USING TWO DIMENSIONAL IMAGES

(71) Applicant: UMOOVE SERVICES LTD., Jerusalem (IL)

(72) Inventors: Yitzchak KEMPINSKI, Geva Binyamin (IL); Sophia FRIJ, Maale- Adumim (IL); Jonathan GUEZ, Jerusalem (IL)

(21) Appl. No.: 14/987,949

(22) Filed: Jan. 5, 2016

Related U.S. Application Data
(63) Continuation-in-part of application No. 14/722,317, filed on May 27, 2015.
Application No. 14/722,317 is a continuation-in-part of application No. 14/723,590, filed on May 28, 2015.
(60) Provisional application No. 62/003,066, filed on May 27, 2014.
(60) Provisional application No. 62/005,045, filed on May 30, 2014.

(51) Int. Cl. G06K 9/00; A61B 3/14; A61B 3/00; A61B 3/113

(52) U.S. Cl. CPC G06K 9/00617; G06K 9/00604; A61B 3/113; A61B 3/14; A61B 3/0025

(57) ABSTRACT

A method and system for detecting patterns of eye movements in a series of captured images may include capturing a series of images of at least one eye of a viewer by a camera, where the series of images may comprise at least two images, calculating, by a processor, a vector of a movement of the at least one eye between the at least two images, where the vector may include a distance of the movement, a direction of the movement and a time between capturing of the images, comparing the vector to at least one characteristic of a plurality of known patterns of eye movements, and classifying the movement of the eye(s) as one of a plurality of known pattern of eye movements.

### CROSS REFERENCE TO RELATED APPLICATIONS

This application is (i) a continuation-in-part of U.S. patent application Ser. No. 14/722,317 filed on May 27, 2015 and entitled SYSTEM AND METHOD FOR DETECTING MICRO EYE MOVEMENTS IN A TWO DIMENSIONAL IMAGE CAPTURED WITH A MOBILE DEVICE, which claimed benefit from U.S. Provisional Application 62/003,066 which was filed on May 27, 2014, and (ii) is a continuation-in-part of U.S. patent application Ser. No. 14/723,590 filed on May 28, 2015 and entitled SYSTEM AND METHOD OF DIAGNOSIS USING GAZE AND EYE TRACKING which benefit from U.S. Provisional Application 62/005,045 which was filed on May 30, 2014. All of the applications listed in this paragraph are incorporated by reference herein in their entirety.

### BACKGROUND OF THE INVENTION

Eye movements under various conditions and stimuli are used as a diagnostic indicator for ailments such as for example neurological conditions. Eye movements however may be extremely rapid, and such movements may not be readily detected or tracked by visual inspection of a medical practitioner. Mechanical or image-based tracking of eye movements has typically required sophisticated laboratory equipment having fixed installations and precise positioning.

### SUMMARY OF THE INVENTION

According to embodiments of the present invention a method of detecting patterns of eye movements in a series of captured images is disclosed. The method may include: capturing a series of images of at least one eye of a viewer, the series comprising at least a first image and a second image, calculating a vector of a movement of the at least one eye between the first frame and the second frame, where the vector may include a distance of the movement, a direction of the movement and a time between capturing of the first frame and the second frame, comparing the vector to at least one characteristic of a plurality of known patterns of eye movements, and classifying the movement of the at least one eye as a first of the plurality of known patterns of eye movements.

According to some embodiments an image of the series of images may be captured using visible light as a two-dimensional image. According to some embodiments, an image of the series of images may be captured with a portable camera included in a portable housing, the housing may include an electronic display suitable for display of a content viewed by the viewer during the capture of the series of the images.

According to some embodiments the distance may be measured as a change in a pixel coordinate of the eye between the first frame and the second frame. According to other embodiments, the pixel coordinate may be measured as a distance of the eye in the first frame from a known object on a face of the viewer in the first frame.

According to some embodiments, the method may further include associating a content viewed by the user during a time of the capturing of the first frame and the second frame, comparing a known pattern of eye movement that is associated with the viewing of the content to the classified first of the plurality of known pattern of eye movements.

According to some embodiments, the method may include identifying a pathology associated with the classified first of the plurality of known patterns of eye movements being different from the known pattern of eye movement that may be associated with the viewing of the content.

In some embodiments, the method may include determining that the movement of the at least one eye between the first frame and the second frame corresponds to either an actual movement of the eye, or to noise.

According to some embodiments, the determining may include scoring an accuracy of a detection of the movement between the first frame and the second frame.

According to some embodiments the method may include determining whether the classified movement of the at least one eye differs from the first of the plurality of known patterns of eye movements, and associating the difference with a pathological condition.

Embodiments of the present invention may include a system for determining an interest of a user in a displayed content, the system may include an electronic display to display a content item, an image capture device positioned at a known distance and orientation from the content item displayed on the electronic display, the image capture device configured to capture during the time period a plurality of images of an eye of a viewer of the content item in a two dimensional image using visible light, a memory; and a processor, the processor to: issue a signal to display the content item on the electronic display during the time period, measure a change in a location of the eye between a first of the plurality of images and a second of the plurality of images; and detect a pattern of the changes in location in the plurality of images.

According to some embodiments the processor may be configured to compare the pattern to a known pattern of eye movements. According to some embodiments the processor may be configured to use the detected pattern to calculate an interest of the user in the content item.

According to embodiments of the invention the memory may be configured to store an association of the user with the content item and the calculated interest.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic drawing of a system in accordance with an embodiment of the invention;

FIG. 2 is a flowchart of a method according to an embodiment of the present invention; and

FIG. 3 is a flow diagram of a method in accordance with an embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention. Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

The processes and functions presented herein are not inherently related to any particular computer, network or other apparatus. Embodiments of the invention described herein are not described with reference to any particular programming language, machine code, etc. It will be appreciated that a variety of programming languages, network systems, protocols or hardware configurations may be used to implement the embodiments of the invention as described herein. In some embodiments, one or more methods of embodiments of the invention may be stored on an article such as a memory device, where such instructions upon execution by for example one or more processors results in a method of an embodiment of the invention. In some embodiments, one or more components of a system may be associated with other components by way of a wired or wireless network. For example one or more memory units and one or more processors may be in separate locations and connected by wired or wireless communications to execute such instructions. Embodiments of the invention may be executed by one or more series of instructions which may be programmed in one or more computer codes, stored as executable code on one or more memories, and executed on one or more processors. Embodiments of the invention may include an article such as a non-transitory computer or processor readable medium, or a computer or processor storage medium, such as for example a memory, a disk drive, or a USB flash memory or other non-volatile memory, encoding, including or storing instructions, e.g., computer-executable instructions, which when executed by a processor or controller, carry out methods disclosed herein.

When used in this document, and in addition to its regular meaning, the term ‘viewer’ may, refer to a person or animal that is looking at or that is situated in a position to look at a display, screen, object, or object shown on a screen.

When used in this document, and in addition to its regular meaning, an eye may include an iris or pupil and may mean or include an area of one or more eyes of a viewer that includes an area of a pupil and iris or such portion of the pupil as may be covered or uncovered in the dilation and constricting of the iris. In some embodiments a differentiation between the iris and the pupil may not be required such that an entire area encompassed by the iris may be included. A capture of an image of an eye may also include a capture of another object on a face such as an image of an eye corner, bridge or tip of a nose, or other feature in an area of an eye(s).

As used in this application, the term ‘fixation’ may refer to a pattern of eye movements. An eye movement pattern may be defined by, for example, direction of movement, speed of movement, and size of movement, during a given time period. Other data may be used. For example if there was no (or close to no) visible or significant movement for a period of time, for example 100 milliseconds or more, it may be assumed that the user is in a fixation eye movement pattern. According to the frame rate a fixation frame threshold may be defined, for example in 30 Frames Per Second (FPS), wherein there are 33.33 milliseconds between frames. If a fixation is detected for a threshold of at least three consecutive frames in which the viewer's eyes did not move (or did not move significantly), it may be assumed that the user is in a fixation eye movement pattern. Other eye movement patterns may include for example saccades, smooth pursuit, micro-saccades and any other pattern known in the art.

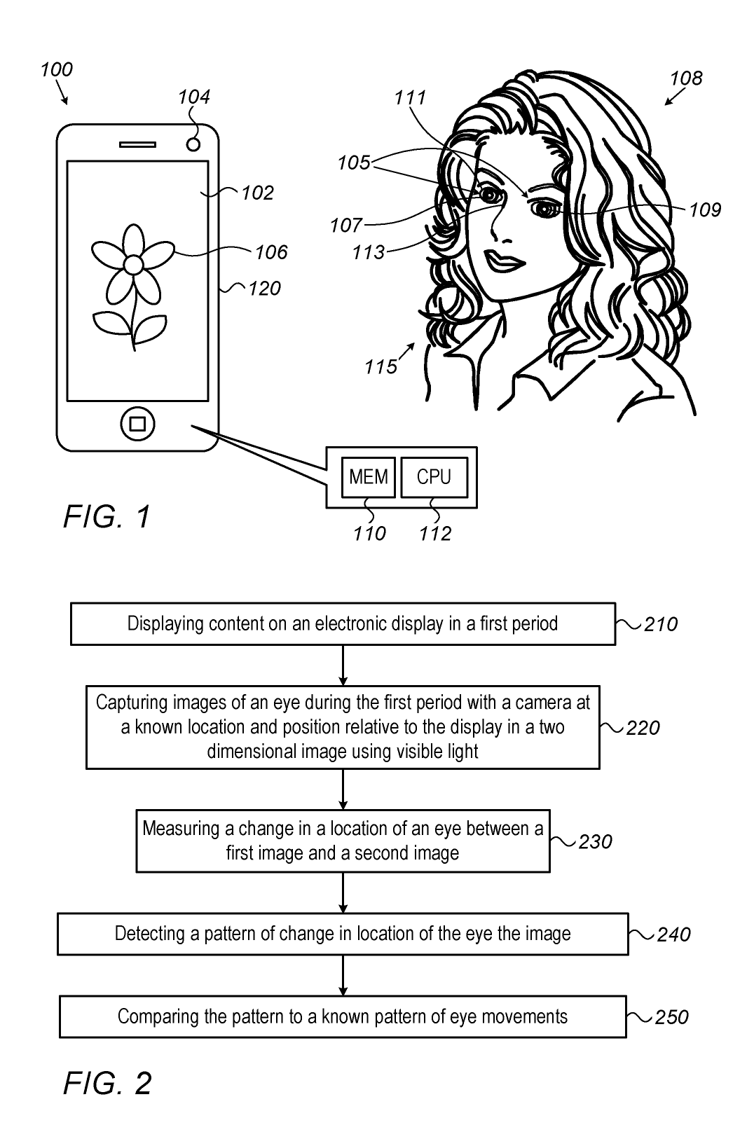

Reference is made to FIG. 1, a diagram of a system in accordance with an embodiment of the invention. System 100 may include an electronic display screen or monitor 102 and a camera 104, imager or image capture sensor. Camera 104 may be at a known distance, location, orientation and/or angle from/relative to screen 102, and from a content 106 displayed on a display or screen 102. Screen 102 may display content 106 such as for example text, images or graphics or other items which may be viewed by a user 108 or viewer. Such content 106 may be still or video and may move or be moved on screen 102. System 100 may be associated with one or more mass data storage memory 110 units and a processor 112. In some embodiments, camera 104 may be or may include an imaging device suitable to capture two-dimensional still or video images using visible light. Camera 104 and screen 102 may be included in a mobile device or housing 120 such as a cellular telephone, tablet computer, laptop computer or other mobile device or a housing thereof. Camera 104 and screen 102 may be in separate housings or devices and may be or be included in a fixed installation such as a desktop computer or work station. Other configurations are possible. A typical frame rate of camera may be 30 FPS though other rates are possible. Camera 104 may capture images of eyes 105 that may include iris(es) 107, pupils 109 as well as portions of a face such as one or more corners 111 of eye 105, bridge 113 of nose or other parts of a face 115.

According to some embodiments, system 100 may include an output device, such as screen 102, or any other output device known in the art. According to one embodiment, output of system 100 may be a diagnosis of a pathological condition of the viewer. For example, aberrant eye movement patterns detected by system 100 may be associated with schizophrenia, and may suggests that the eye movement testing has efficacy as a test of gene carrier status in schizophrenia, and therefore as a trait marker of risk for schizophrenia, as further described herein. According to some embodiments, the output of system 100 may be a signal or an indication of the engagement and interest level of the viewer with content 106 presented on display or screen 102 as further described herein.

Reference is now made to FIG. 2, which is a flowchart of a method of determining an eye movement pattern according to some embodiments of the present invention. As seen in block 210 content, such as content 106 in FIG. 1, may be displayed on a screen such as screen 102, or any other electronic display, for a first period. Content 106 may be any object presented on screen 102 such as a video, animation or graphics displayed to the user on one or more portions of screen 102, for example, during one or more time intervals.

According to some embodiments, in block 220 imager or camera, such as camera 104 in FIG. 1, may capture a series of images of one or both eye(s) 105 of a person, and particularly of the iris(es) 107 and/or pupil(s) 109 of the person 108. Camera 104 may capture a series of images of at least one eye of a viewer, where the series of images includes at least a first and second image. The series of images may be a time-ordered sequence of images, a video, etc. One or more filtering processes may be implemented on one or more of the series of images to minimize, discount or eliminate the effect of inaccurate or unreliable images in the series (referred to in this application as ‘noise’), disturbances affecting the eyes, poor lighting or poorly captured images in the series of images.

According to some embodiments, a method or processor 112 may measure changes in a location of an eye or a portion thereof (e.g., iris 107, pupil 109 etc.) between a first image and a second image or any other set of consecutive or non-consecutive images, and detect and/or calculate positions or movement patterns of eye as appearing in the captured series of images (blocks 230 and 240). A vector may be calculated of a movement of at least one eye between the first captured image frame and the second captured image frame. The vector may include a distance of the movement of the eye in the two images, a direction of the movement and a time elapsed between the first frame and the second frame (e.g., the amount of time between the capture of the first frame and the capture of the second frame), and patterns of such movements of eye 105 may be associated with content 106 shown on screen 102 or otherwise seen by the viewer at a time of the capture of the images.

As seen in block 250, the patterns of such movements of eye 105 may be classified or designated as being of one or more known patterns of eye movements. A comparison may be made between the calculated vector and at least one characteristic of one of two or more known patterns of eye movements. The vector or series of movements as were detected in the image pair may be divided into various characteristics including speed of movement, frequency of movement, distance of movement and others. The presence and/or values of one or more of these characteristics may be compared to characteristics of known patterns of eye movements. The pattern of eye movements that was detected in the images may be classified as being or being similar to, or as being, one or more of the known patterns of eye movements, based on that comparison. For example, a vector or series of movements that were detected in pairs of images may indicate that the viewer's eyes are moving in a slow and constant speed in a specific direction so that the speed and direction correlate with the speed and direction of movement of the object displayed on the screen. These eye movement parameters or characteristics may be compared to stored parameters or characteristics of known patterns of movement and based on the comparison it may be determined that, for example, the captured movement of the eyes is similar to or may be classified as, for example, smooth pursuit pattern.

In some embodiments, the images may be captured using visible light as a two-dimensional image. In some embodiments, images may be captured with a camera included in a portable housing that has an electronic display suitable for display of a content viewed by the viewer during the capture of the images.

In some embodiments, the distance may be measured in a change in one or more pixel coordinates of a location of an eye between a first frame and a second frame. In some embodiments, a pixel coordinate may be measured as a distance of the eye in the first frame from another object on a face of the viewer as such object appears in the first frame and/or in the second frame.

In some embodiments, a content or a movement or action of a content appearing on a screen, that is viewed by the user when the first frame and the second frame were captured may be associated with a known pattern of eye movements. A comparison may be made between the known pattern of eye movement that is associated with the viewing of the content to the classification that was determined as matching the detected pattern of eye movements in the images.

In some embodiments, upon an observation of a difference between the classified eye movement pattern that was detected from the captured image, and the expected eye movement pattern that is associated with the content shown to the viewer, a pathology may be identified as associated with such difference.

Embodiments of the invention may include determining that one or more of the detected eye movement between the first frame and the second frame corresponds to either an actual movement of the eye, or to falsely detected, irrelevant movements, noise or other non-reliable detections of movements.

In some embodiments, an accuracy or reliability score may be applied, attributed or assigned to one or more detected eye movements in one or more frames.

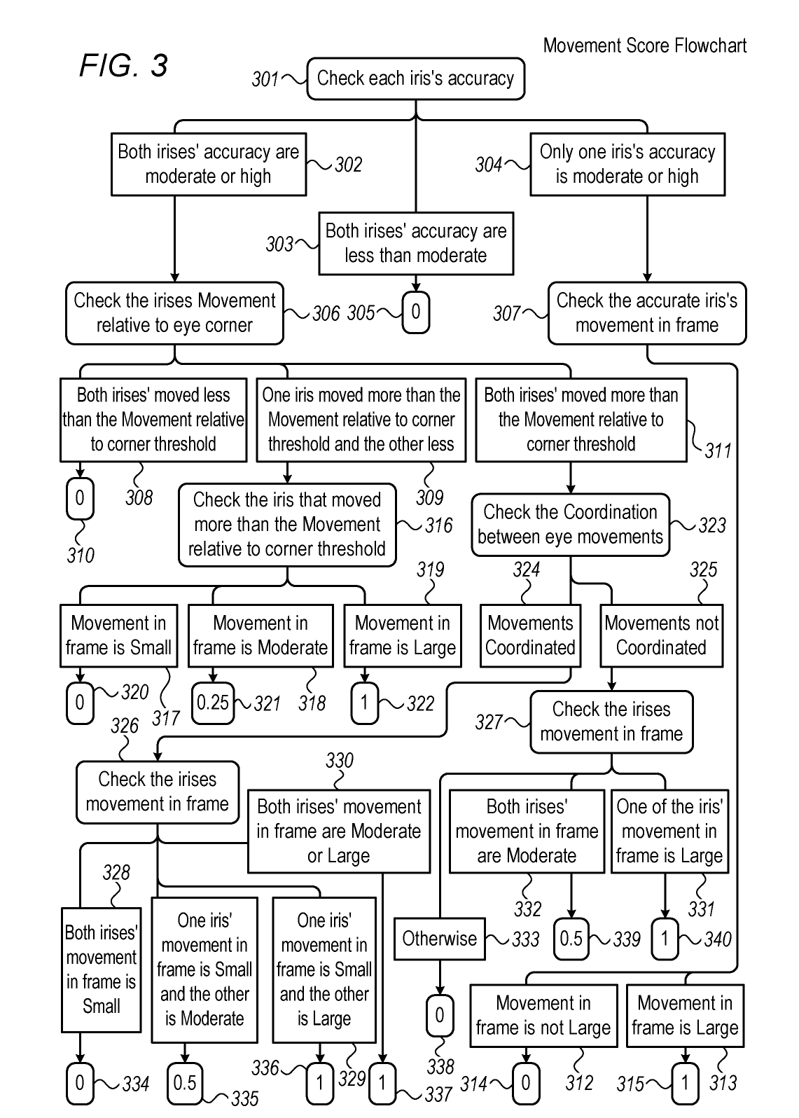

As further detailed with reference to FIG. 3, a reliability of the detected and characterized patterns may be calculated. The detected pattern may be correlated with the state of the viewer or the activities undertaken by the viewer or the content 106 that the viewer was observing at the time that the images were captured. The detected patterns may be compared with patterns that are expected from a person in such state or when viewing such objects. Deviations of the observed patterns from the detected patterns may be included as inputs in a diagnosis of a person.

According to some embodiments, patterns of eye movements may be detected in, during or between two or more or a series of frames. Such patterns may be classified as one or more of saccades, smooth pursuit, micro-saccades and fixation, or as other patterns. One or more characteristics of such patterns may be known and may be stored for example in a memory associated with system 100. A time or period of time of a detection of a pattern of an eye movement by a viewer may be recorded, and correlated with a time of an appearance on a screen 102 viewed by the viewer 108 of an object or content 106 or the time of viewing by the viewer of some other object. According to some embodiments, a state of mind, level of interest, level of attention, period of attention or other characteristic of a viewer's interest in or observance of the content 106 or object that appeared on the screen 102 may be determined, calculated, implied, estimated or assumed from an eye-movement pattern detected at a time the object 106 appeared on screen 102 or elsewhere in view of the viewer or at the time that the viewer 108 was in some other state of mind or undertaking some other activity.

In some embodiments, a movement of an eye 105, iris 107 or pupil 109 relative to another object on a face such as an eye corner 111, bridge 113 of nose, tip of nose or other body part or object at a known orientation to an eye or iris may be calculated. The eye movement relative to the eye corner may be calculated for one or both of an X and Y axis (e.g. of a Cartesian coordinate system) of for example pixel positions in one or more of the captures images. For example, if in frame A, iris location in pixel position coordinates is (100,60) and an eye corner location is (123,52), and in frame B iris location is (105,57) and eye corner location is (120,53), then the Iris/Eye Corner Movement of Frame A-B may be for example: X=abs(100-123)-abs(105-120)=8, Y=abs(60-52)-abs(57-53)=4. Iris/Eye movements may be measured from other visible portions of a face or known body parts such as a bridge of a nose, point of nose, or other body parts that are seen in the captured image. Values of movements may be measured across numerous consecutive (A-B, B-C) or non-consecutive (A-C, B-D) frames. Each of X and Y coordinate movements across frames (movements indicated by a change in position of the eye or pupil from one frame to another frame) may be positive or negative to indicate a direction of movement, and one or more of a series of vectors of frame movements may be established. If an image capture speed (usually denoted in frames per second ‘FPS’) of an imager is known, a speed of movements of an eye may be calculated and associated with a vector or direction of movements of the eye over a period of capture of two or more frames. A series of movements or movement vectors that may include a direction, distance/magnitude and a speed of such movements between or among two or more of such captured frames may be collected.

Detected movements may be categorized into for example three or more predefined levels, such as small movement in or in between frames, below which the movement is assumed to be noise; moderate movement between frames, such that the detected movement may be noise or actual eye movement; and large movement between frames, where the movement is assumed to be actual eye movement. Other categorizations or number of categorizations may be used. Thresholds or categories of iris versus eye-corner movement may also be categorized, such that movement distances below a certain threshold may be assumed, weakly assumed or strongly assumed to be noise, and movements above one or more thresholds may be assumed, weakly assumed or strongly assumed to be or indicate actual movement.

Use of standard filters to reduce noise from the captured images can be problematic because eye movement may change rapidly, for example from smooth small movements to very high speed movements. In some embodiments, detected movement of eyes or pupils between or among captured frames may be filtered or subject to one or more filtering processes to distinguish between actual or relevant movements on the one hand, and noise or other unreliable or untrue movements on the other hand, that may be detected in the images or between frame pairs. In some embodiments, a probability may be calculated for a reliability of a detected eye movement as to whether a detected movement among images is actual and relevant or noise and therefore less relevant.

Though various image filtering processes may be employed, the speed of eye movement relative to standard frame capture rates may present the following filters with an advantage for detecting patterns of eye movements.

When there is a detected eye movement that is suspicious because of a prior or expected movement, an appropriate filtering procedure may be to wait for a position or movement between two or more subsequent frames in order to confirm or discount the movement detected between prior frames that was not the expected movement. For example, if an eye movement between Frame1 (F1) and Frame2 (F2) was consistent with a fixation pattern, and a movement between F2 and F3 was consistent with a saccadic pattern, a resolution or filtering of the unexpected pattern in F2-F3 may include waiting for movement of F3-F4 or some other subsequent frame series, and detecting a movement in such later series of frames to confirm or discount (or improve a probability of confirming or denying), the suspicious or unexpected movement that was detected in F2-F3.

In other examples, under usual circumstances a working assumption may be that a detected pattern of eye movements between a first set of frames is likely to continue in an immediately subsequent set of frames. Such patterns of eye movements may include for example: Fixation—eyes maintain a visual gaze on a single location, so the irises should be found in a single location (or very close to that single location) in a consecutive or nearly consecutive series of frames. Saccade—rapid, ballistic movement of the eyes that abruptly change the point of gaze, where the irises should be detected to have moved more than a minimum and less than a maximum number of pixels in the image of the eye socket or relative to the eye corner between two or more frames or series or frame pairs. Smooth pursuit—Eyes move smoothly following an object, where the irises should be detected to have moved an equal (or close to equal) distance in a series of frame pairs, and more than a minimum number of pixels between each of such frame pairs, or where the irises should be found to have moved more than a minimum number of pixels between frame pairs but at a low acceleration. Micro Saccade—small eye movements, where the irises should be found to have moved less than saccade movements as described above, but more than a minimum number of pixels in the eye socket between frame pairs. The minimum number of pixels described above, may be a number of pixels that is generally associated with no appreciable eye movement between frame pairs.

In an embodiment of this filtering procedure, if in a first frame pair or first series of frame pair a micro saccade movement is detected, a detection in a subsequent frame pair of a fixation pattern may be unexpected or suspicious. A determination of whether the unexpected movement is relevant or noise may be reached by comparing the suspicious movement to a movement in a subsequent frame pair. The detected pattern in such subsequent frame pair may confirm, reject or discount the relevance of the suspicious movement or pattern.

In another scenario, if a location of an iris has not moved from a specified location or area in a last group of frames, a found location that is out of such area may be dismissed until another detected location in further frames confirms the iris being out of that area.

Another procedure for filtering noise or non-relevant eye movements from one or more frames may include assigning a reliability score to the eye tracking data, and altering the weight given to a detected movement using the reliability score. For example, detecting a position or movement of an eye may proceed in stages, where one or more of such stage gives additional confirmation or information about the iris's location and the relative accuracy of the iris detection. In some embodiments, the more stages that the location passes, the higher becomes the reliability of the detected location. A detected location of an iris that seems un-reliable may be dismissed, discounted or changed according to refined or improved location detections in subsequent frames or according to the movement of the other iris (assuming that both irises of the individual move in unison). In some embodiments, the detection stages may include one or more of the following (order of the stages may be varied): 1) Values—comparing pixel average lightness values of a detected iris to previous values or know range of values for previously detected irises. 2) Generalized Shape—comparing a detected iris in a frame or series of frames to a generalized or assumed iris shape, such as a circle that is dark on the inside, and bright on the outside. 3) Detailed shape—Finding pixel edges in close range of detected iris, and adjusting a circle to the detected pixel edges using for example a weighted least squares method. Reliability points may be scored based on the distance of pixels from the adjusted circle using a threshold to determine the edge value of the detected pixels relative to the position of the adjusted circle. 4) Correlation and position—Measuring an angle between eyes, calculating a distance and position of the irises relative to the eye corners and comparing some or all of the position, angles and distance of the detected iris relative to the eye corners or other face points in an image. A determination may be made as to whether the detected position of the irises fits an expected position, distance or angle to the eye corners.

With reference to FIG. 3, which is a flowchart of a method of scoring eye movements, as seen in block 301, upon application of one or more of the detection or filtering stages listed above, an accuracy or reliability rating or score may be calculated and applied to the iris or eye that is detected in one or more images. For example, a reliability score may be calculated for a detection of an iris in an image, and scores may be applied to the image, such as for example −1,0,1,2,3, where −1 is a bad, unreliable or probably inaccurate result; 0 discounts the detection completely; 1 represents an undetermined accuracy probability; 2 represents a moderate accuracy probability; and 3 represents a high accuracy probability. Scores may serve as a weighting of a reliability of eye position detections in one or more images. Such scoring may be part of a decision of whether to rely and to what extent on the detected iris location. In some embodiments, application of a reliability score may limit the importance of ‘noise’ or false detections in an image by minimizing the importance or weighting of non-relevant, poor or false detections of an eye in an image. Scores, points, etc., discussed herein have specific examples and ranges provided herein, but in other embodiments other ranges and values may be used.

A further filtering process may track both eyes of a viewer, and may prefer one eye over the other or may balance between the results of the two trackings. Tracking two eyes is of course relevant where the eyes are being tracked together, and not in order to compare their movement correlation. For example a filtering process may apply a reliability score as was described above, and may use a tracking of a first eye when the reliability score of such detected eye position was higher than the reliability score applied to the tracking of the other eye. A filtering process may average the reliability of the detection of both eyes, or reduce the relevance of a first eye if it detects that the second eye is following a naturally expected pattern (based on for example previous frames) while the first eye is doing an unexpected movement.

As seen in blocks 302, 303 and 304, the accuracy of the irises or other portion of the eyes may be determined to be either moderate or high for both eyes, less than moderate for both eyes or only one iris's accuracy is determined to be moderate or high.

When both irises' accuracy is less than moderate the score that is assigned to the movement may be for example 0 (block 305).

As seen in block 307, according to some embodiments, when only one iris's accuracy is determined to be moderate or high, the movement of the accurate iris may be measured. When the measured movement of the accurate iris is large the score that may be assigned to the movement may be, for example, 1 (blocks 313 and 315) while when the movement of the accurate iris is not large the assigned score may be 0.

Thresholds or categories of iris to eye corner movement may also be applied as a filter by for example categorizing eye movements relative to eye corners that are above or below a certain fixed or dynamically adjusted threshold. For example, movement distances between one or more frame pairs that are below a certain threshold may be assumed, weakly assumed or strongly assumed to be noise (blocks 308 and 310), and eye movements between one or more frame pairs that are above one or more thresholds may be assumed, weakly assumed or strongly assumed to be or indicate actual movement (blocks 309, 316, 317, 318, 319, 320, 321 and 322).

As seen in block 311, according to some embodiments, when both irises or other portions of the eye, move more than the threshold, relative to the movement of the eye corner, a correlation between eye movements of both eyes may be checked (block 323).

A further noise filter may rely on an assumption that the irises of a person usually move together or in coordination. Analyzing or comparing detected movements of a person's two irises relative to each other may assist in verifying whether the detected movement is an actual movement or noise. Iris movement coordination may be examined by calculating each iris's motion vector. Two or more images of one or more eyes, irises and/or areas around an eye may be captured by camera 104. A location or coordinates (such as pixel coordinates in the frame pairs) of one or both eyes or irises in each of the frames may be determined. A comparison may be made between a location or coordinates of one or each of the irises in a first, previous or prior frame and a location or coordinate of the same iris in a second, subsequent or later frame. A first eye vector may be calculated between the location or coordinate of a first iris in a first frame and a location or coordinate of the first iris in a second frame. A second eye vector may be calculated between the location or coordinate of a second iris in a first frame and such second iris in a second frame. An angle may be calculated between the first iris vector and the second iris vector. Using the assumption that irises move together or in coordination, a small angle such as below 90° (though other thresholds may be used) between the first iris vector and the second iris vector may be an indication that the two eyes moved together or in coordination. If the angle is large, it may be an indication that the eyes did not move together or in coordination, and that there is either a physiological problem with the viewer or that there is noise in the detection. Noise movements may be eliminated or have their significance reduced. As seen in blocks 324-340, the correlation between movements of each iris or between iris movement vectors of both eyes, and the angle or size of movement determine the score that would be assigned to each movement.

Patterns of eye movements may be detected in or between two or a series of frames, and such patterns may be classified as one or more of saccades, smooth pursuit, micro-saccades and fixation, or other patterns. A time or period of time of a detection of a pattern of an eye movement by a user may be recorded, and correlated with a time of an appearance on a screen 102 viewed by the user 108 of an object or content 106. A state of mind, level of interest, level of attention, period of attention or other characteristics of a user's interest in the content object that appeared on the screen 102 may be determined, calculated, implied, estimated or assumed from an eye-movement pattern detected at a time the object 106 appeared on screen 102.

When an eye movement has been detected and filtered for unreliable detection or noise, the detected eye movement (measured relative to for example, the eye corner/eye socket/face) in a specified frame or small group of frames may be classified to specify the type of eye movement the eye (or eyes) is performing. Detected movements of eyes in two or more frames may be determined, subjected to a ranking for confidence of actual or significant movements as opposed to noise, and categorized or classified into known or recognized patterns of eye movements.

A list or collection of expected, frequent or normal eye movement patterns may be designated or stored, and characteristics of such movements may be stored. For example, as described herein, eye movements may be found in pattern of any of fixation, saccade, smooth pursuit, micro-saccade or others. A detected iris movement may be analyzed to determine a closest or best match of the detected movement with a known pattern. One or more basis of comparisons may be used such as analyzing a size or magnitude of the detected movement in one or more frame pairs, analyzing the speed/acceleration of the movement, analyzing the direction of the movement relative to previous movements.

By way of example, analyzing the size of the detected movement may include determining that if the eye movement is large, such as larger than a pre-defined threshold, the movement may be consistent with one or more of a saccade, a micro-saccade or part of a smooth pursuit movement. To further narrow the classification possibilities of the detected movement, the acceleration of the movement may be determined using for example the following formula: Acceleration=Current Movement-Previous Movement.

If the acceleration is lower than a predefined threshold the movement may be classified as smooth, and the direction of the smooth pursuit may be the direction of the movement. If the acceleration is high, the movement is likely not smooth, and the classification possibilities may be narrowed to saccade or micro saccade. In saccade, the movements are large ballistic movements, so if the movement is larger than a predefined saccade threshold, the movement may be classified as a saccade. Otherwise the movement may be classified as a micro saccade. Other ways of selecting or matching a known movement pattern to observed movements are possible.

If the detected movement is not large, the pattern may be classified as one of a slow smooth pursuit movement or a fixation. To further narrow the classification, an analysis may be made of the movement and the direction of the movement. A movement that is slow and smooth may be difficult to detect at slow frame rates such as 30 fps, so it may be helpful to evaluate movements over a longer series of frames rather than just a frame pair. For example, a sum of detected movements over for example eight frame pairs or movement detections may be calculated, and a vector of such movements may be determined using for example the Pythagoras theorem or in another way. An x-axis movement direction may be calculated as the sum of movement of the last 8 frames on the x axis divided by 8. The y axis movement direction may be calculated as the sum of movement of the last 8 frames on the y axis divided by 8. Other numbers of frames may be used.

If the movement vector is larger than a predefined threshold, the summing may be performed again for example by: remove or discount large movements from the movement vector since they may be noise movements if the movement is a slow-smooth; subtract opposite direction values from the largest movements (if the x axis direction is right, subtract all movements that go left etc.); if one of the movements is still larger than for example 70% of the total sum, add it as an 8th of a minimum smooth predefined threshold; check if the movement vector is still larger than a predefined threshold, if so, the movement may be classified as smooth, and the direction of each axis may be the direction of the smooth pursuit. If the movement is not a slow smooth, then the movement may be classified as fixation. Other methods and processes for classifying eye movements into known patterns may be used.

Smooth pursuit: If the score as determined in accordance with FIG. 3 is not zero, or is otherwise indicative of an eye movement pattern, the eye locations may be examined further for smooth pursuit movement looking for some or all of the following patterns or characteristics: Speed of movement between frames is less than a predetermined threshold for example if the user's eye moved less than 0.2 centimeters (cm) since the last frame (where 0.2 cm is the predetermined threshold for the current frames per second (fps)), then the type of movement may be smooth pursuit. Smooth pursuit may further be identified if there is a small angle between the current eye movement vector and a previous eye movement vector. Smooth pursuit may also be present if current and previous eye movement vectors create a relatively smooth pattern of changes in speed and acceleration.

A micro saccades pattern may be identified if real eye movement (rather than noise or other immaterial movement) is found and real space movement is less than a small predefined threshold (such as less than 2 degrees).

A saccades pattern may be detected if real eye movement is larger than a predefined threshold.

In some embodiments, eye movements between two or some other small number of frames may be determined or collected. A further, additional or alternative analysis may be made of a sequence of frames that occurred in a period of time. An estimate may be made of the activity in the specified time period summing up the amount of saccadic movements, fixations and smooth pursuit that occurred. A conversion may be made of the movements into percentages of the time period under analysis (for example one second) taking into account the current FPS. The result may be the basis of analyzing the user's state of mind during that period of time. For example a high amount of saccadic activity with repeated fixations/smooth pursuits throughout the time period may indicate a higher level of engagement and interest.

In some embodiments, it may be desirable to correlate a detected and classified eye movement with a state or behavior of the viewer, or an activity or view seen by the viewer at the time of such movement. In some embodiments a predicted, assumed or expected eye movement may be associated with a state, activity or view seen by the viewer at the time that the pattern was detected. This may be defined based on the specific application or goal of the viewing test, and the knowledge of what is being presented to the user, what he is being asked to do and what are expected and possible responses. One or more actions, states or behaviors may be analyzed to determine the types of eye movements that may occur or be expected to occur while the viewer is performing the action. Thus the eye movements may be examined over time, and divided into states of action. Lists of possible viewer actions may be classified, collected and examined with respect to the eye movements that were expected. For example, during staring, a viewer is looking at one point; during looking at or looking away, a viewer may be prompted to look at a single point and then promoted to look away from such point. Movement may be as a response to stimuli appearing or changing or in response to a sound or voice commands. When a viewer is following an object, the viewer may be watching a moving object and following it with his eyes. For testing a level of engagement in the content, an evaluation may be made of the viewer's interest in or engagement in the content he is viewing. In some embodiments, such engagement may be determined by the amount of rapid scanning of the content.

A further, additional or alternative analysis may be made of a sequence of frames that occurred in a period of time. An estimate may be made of the activity in the specified time period summing up the amount of saccadic movements, fixations and smooth pursuit that occurred. A conversion may be made of the movements into percentages of the time period under analysis (for example one second) taking into account the current FPS. The result may be the basis of analyzing the user's state of mind during that period of time. For example a high amount of saccadic activity with repeated fixations/smooth pursuits throughout the time period may indicate a higher level of engagement and interest.

Actions may also include unconscious behaviors such as eye correlation of the two iris movements, and may correspond to an expected performance such as following a moving object or a set of instructions; level of jumpiness in the eye (nystagmus); or blink patterns categorized by frequency of blinks as well as their duration. Other actions or patterns may be detected.

Once a category of action or state of mind is defined, eye movement patterns may be collected and associated with such defined actions to determine the types of eye movements that may occur while the viewer is performing the action. For example, when staring, eyes may be fixated on one point. If the head moves and the eyes are still staring at a point, eye movement may be determined as smooth pursuit in the opposite direction of the head movement and with the head movement's speed. If the object being stared at moves and eyes are still staring at a point, the eye movement may be determined as smooth pursuit to the direction of the movement and with more or less the same movement's speed or estimated/expected speed. In some cases, as long as there is no saccade from the time the user begins to look at the object being stared at, then he may be said not to have looked away. Other measures may be used as validation that the images did not miss the user's looking away.

Further, when a user is looking at content displayed on a screen or away from the content displayed on the screen, the eyes may move in a ballistic way via a saccade from a first location to another location. Movement may occur as a response to stimuli appearing or changing, or in response to a sound or voice command. For example if an object was displayed, and a ballistic-saccade eye movement was detected in the direction of the object, the viewer is most likely looking at the object. With respect to following a moving object, the eyes move in a smooth pursuit more or less in accordance (with respect to velocity and direction) with the moving object on a screen. If the movement is slow it can be expected that several fixations will be observed during the process. If the fixations are not extreme and the classification keeps returning to smooth pursuit, then the user is probably still following because otherwise a saccade would be observed. Another indication of a viewer following an object can be head movements that generally correlate with the direction and/or speed of a moving object while the eyes may seem to be fixated.

Indications of level of engagement in the content may include the eyes being fixated and moving from one point to another on the observed content. A level of engagement may be determined by the amount and/or frequency of such movements. Counting these movements per second may provide an estimate or scale of the engagement.

An embodiment of the invention may evaluate the correlation of the eyes of a viewer by for example analyzing each eye individually in groups of frames, and comparing the activities of the two eyes to each other to determine whether they move in a coordinated manner. This may be done while a viewer is following a moving object, following a set of instructions or in response to other stimulus. The groups of frames may be analyzed to determine whether the eye movements are correlated or not correlated with a pattern and with an action or state of mind at the time that such pattern was observed, and may evaluate the correlation when the viewer is viewing various objects or performing certain tasks.

Embodiments of the invention may be used to evaluate a level of jumpiness in the movements of eyes (nystagmus). The eyes of some people may exhibit jumpy movements while is staring at something. An embodiment may detect that in certain series of frames there are sets of frames in which the irises move gradually via saccades or micro saccade movements or smooth pursuit movements, and proceed with a frame or a few frames with the same type of movement to the opposite direction. A detection of this pattern may indicate that a viewer has jumpiness in the eye or some related condition.

In some embodiments it may be beneficial to verify a quality of the detection of patterns and the tracking of movement of the eyes in one or more series of frames. Many variables and environmental conditions may affect eye tracking, and a status assessment may estimate whether the tracking is stable enough to be relied upon. This may be especially important if an embodiment of the system is used for medical or diagnostic purposes. The iris tracking quality may be assessed over a period of time such as for example 2-3 seconds and in various ways, that take into account different factors that may indicate possible problems. For example, eye movements may be checked for data that indicates movements that seem unnatural or not physically possible, such as movements that are too large or/and fast or too many/unnatural direction changes in the movements of the eye. For example, a check for reliability may include counting the number and magnitude of direction changes in a specified period. For example, if in a designated number of seconds, such as two seconds, a normal eye may undergo not more than a maximum number of direction changes, then a detected number of direction changes over such period may indicate that the reliability of the detection is suspect. A detected number of changes that is lower than the maximum may be assigned a satisfactory or non-suspect stability quality. In some embodiments, a stability, quality or reliability rating may decrease in proportion to the excess of the detected number of direction changes over the known maximum number of direction changes.

A further reliability rating may use the detected or obtained locations of the eyes in one or more frames. If there are many frames with uncertainties or that show that the eyes are detected in locations that are impossible or inconsistent with known face data or other known factors, the tracking may be faulty and a reliability rating may be decreased to indicate the lower the overall tracking quality. Such reliability ratings may be tracked or evaluated on a continuous, sporadic other basis and may be initiated in relation to a specific expected task or behavior that is being observed. For example, a quality rating may be implemented continuously during a neurologic exam, but sporadically when evaluating an engagement of a user with a television commercial. In some embodiments a quality or reliability rating may be set with one or more thresholds depending on an application's need and the level of accuracy and resolution required for the specific evaluation being performed. For example, an evaluation of possible nystagmus may require a high reliability threshold, while a lower threshold may be tolerated for an attention deficit evaluation.

In some embodiments, a deviation of a detected pattern from an expected pattern may be evidence of one or more pathologies. In some embodiments, a processor may collect data about at least one gaze point of a viewer at an object appearing in a displayed image as the viewer looks at the display in the series of image. The processor may compare the data about the person's gaze in such images with stored gaze data of persons who may have a pathologic condition. Such conditions may be included or be selected from the group consisting of schizophrenia, attention deficit-hyperactive disorder, amyotrophic lateral sclerosis, concussion (mild Traumatic brain injury mTBI) or Post Concussive Syndrome (PCS), autism, optikinetic nystagmus and Alzheimer's Disease. Eye and gaze tracking may also be used in the diagnosis and/or treatment and management of diseases including vestibular disease and vertigo.

For example, Alzheimer's Disease (AD) is associated with deficits in visuospatial cognition. Neurophysiological and imaging studies have revealed that changes in visuospatial perception (VSP) functions can be detected in the early stages of AD. Key advantages of utilizing VSP-related deficits in AD for early detection and longitudinal tracking of AD. AD patients may need significantly more time to initiate and execute goal-directed hand movements. AD patients are also unable to suppress reflexive eye and, to a lesser extent, hand movements. Furthermore, AD patients use a stepwise approach of eye and hand movements to touch a sequence of stimuli, whereas controls more often show an anticipatory approach. The impairments in reflex suppression of eye and hand movements, and changes in relative timing of eye-hand coordination, in AD patients support the notion that cortical networks involving the posterior parietal cortex are affected at an early disease-stage. It also suggests that the problems of AD patients in performing daily activities that require eye-hand coordination are not only caused by cognitive decline, but also by degeneration of neural networks involved in visuomotor coordination. Some or all of such indications may be observed in eye movement patterns that are detected in a series of images captured and analyzed in accordance with an embodiment of the invention and the automatic detection of cognitive impairment using on eye tracking data. Visual Paired Comparisons such as saccade orientation, re-fixations and fixation duration, and other saccade dysfunction may also be associated with cognitive decline maladies such as Alzheimer's disease.

Aberrations in some or all of the following patterns may be associated with a diagnosis of autism, looking, gazing, fixating, (stabilized gaze on fixed target) visual attention, eye movements, saccades, and smooth pursuit, peripheral cues, saccadic latencies, oculomotor performance, visual orienting, action predicting and gaze following, predictive tracking. Abnormal facial scanning may be associated with a diagnosis of autism.

Saccadic eye movement abnormalities, such as a decline in prosaccade latency and velocity may be associated with Huntington's disease.

Detection of concussion, mild Traumatic brain injury mTBI, Post Concussive Syndrome PCS may be assisted by detection of aberrations in anti-saccades tasks that rely on discrete stimulus-response sets. In some embodiments analysis of anti-saccades tasks may be useful once subjects have perceived PCS symptoms. Because attention varies over time, a relatively continuous measure of performance is needed to detect moment-to-moment fluctuations in attention within individuals. The examination of performance of visual tracking of a moving target may provide a supplement to conventional behavioral assessments of mTBI patients. Using video-oculography, eye movement can be monitored easily, precisely, and continuously. In contrast to the anti-saccade paradigm, visual tracking does not rely on discrete stimulus response sets during the maintenance phase. Visual tracking of a moving target may require the integration of multiple sensory inputs and the subject's own motor efforts. Visual tracking also requires cognitive processes including target selection, sustenance of attention, spatiotemporal memory, and expectation. Quantification of visual tracking performance using a circular target trajectory, provides a continuous behavioral assessment metric. The motion of a target traveling at a constant velocity with a fixed radius from the center is highly predictable. This movement can continue indefinitely within the orbital range of the eye, which makes the stimulus particularly suitable for studying the processes required to maintain predictive visual tracking. Predictive visual tracking requires both attention and working memory, processes for which the PFC is considered to be an important substrate. These cognitive functions are often compromised in mTBI patients. Visual tracking performance can be objectively quantified using parameters, such as smooth pursuit velocity gain, phase error, and root-mean square error. Poor visual tracking in mTBI subjects is generally characterized by a wide distribution of the gaze along the circular path, which indicates spatio-temporal dyssynchrony with the stimulus. The spread of visual-tracking gaze errors (variability) can be used as an attention metric and can be correlated with an individual's degree of white matter integrity. Frequent lapses in attention area characteristic of Traumatic Brain Injury (TBI) Anti-Saccades tasks, a type of eye movement paradigm sensitive to frontal lobe dysfunction that may rely on discrete stimulus-response sets. Anti-Saccades tasks may be useful in detecting Post-Concussion Syndrome (PCS). Visual Tracking performance may provide a continuous behavioral assessment metric and is highly predictable, and often compromised in mild traumatic brain injury.

Post-Concussion Syndrome (PCS) patients may perform badly on anti-saccades, self-paced saccades, memory-guided sequences and smooth pursuit tasks. Eye movement function in PCS does not follow the normal recovery path of eye movements after mCHI, marking ongoing cerebral impairment independently of patient self-report and neuropsychological assessment. Eye movements may provide evidence of dysfunction in areas such as decision making under time pressure, response inhibition, short-term spatial memory, motor-sequence programming and execution, visuospatial processing and integration, visual attention and subcortical brain function. Distorted eye movements may be of particular interest in PCS cases with high symptom load and poor ability to cope with activities of daily living but whose clinical test profile is otherwise unremarkable with regard to neuropsychological testing or other assessments. Eye movement testing, and evidence of suboptimal subcortical functioning in particular, may help demonstrate incomplete recovery of brain function in such cases.

Convergence insufficiency is often associated with symptoms such as frequent loss of place while reading, loss of concentration, having to re-read, reading slowly, poor comprehension, sleepiness, blurred vision, diplopia, headaches, and/or eyestrain. Detection of eye patterns corresponding to such complaints may allow early diagnosis of these conditions. Further computer accommodative therapy using eye movement pattern detection may allow in-home monitoring of the condition and improvement resulting from exercise therapy.

Aberrant eye movement patterns may be associated with schizophrenia, and may suggests that the eye movement testing has efficacy as a test of gene carrier status in schizophrenia, and therefore as a trait marker of risk for schizophrenia.

Impaired smooth pursuit eye movements may be an indicator in unaffected relatives of psychiatric patients, and may be important in excluding non-specific effects (e.g. medication) and isolating genetic predisposition to such diseases. Pathological distributions of various parameters of smooth pursuit eye movement may be observed in groups of schizophrenic patients and their relatives. Eye tracking dysfunction may be associated with two gene polymorphisms that interfere with dopamine metabolism and are thus reasonable candidate genes for the predisposition to schizophrenia.

Observing slowing saccades, decreased smooth pursuit, and decreased velocity may be associated with pharmacological effects of Benzodiazepenes, antipsychotics. Increase in peak velocity of prosaccades may be associated with an side effect of anti-depressants. Shortened latency may be associated with nicotine exposure or addiction.

Eye movement recordings may be a useful addition to the clinical examination. Laboratory recordings of eye movements may provide valuable information about disease severity, progression or regression in neurodegenerative disease, and hold particular promise for objective evaluation of the efficacy of putative neuroprotective and neuro-restorative therapies. Ailments that may present or be detected by tracking of eye movements include Parkinson's disease, Glaucoma, Fragile X Syndrome, Progressive Supranuclear Palsy, Attention, and Affective Disorder. Abnormal Smooth is an observed neurophysiologic deficit in schizophrenia patients, and Parkinson's disease. Hypometria type abnormality for vertical saccades may be observed in patients with Parkinson's disease.

Processor 112 may be, for example, a central processing unit processor (CPU), a chip or any suitable computing or computational device. Processor 112 may be configured to carry out methods described herein, and/or to execute or act as various modules, units, etc. Processor 112 may include more than one CPU or other computing device. In one embodiment, by executing executable code or software for example stored in memory 110, processor 112 may be configured to carry out methods as described herein.

Embodiments of the invention may include a method or system for determining an interest of a user in a displayed content. Such a system may include (or include the use of) an electronic display to display a content item during a predefined time period, an image capture device such as a camera that is at a known distance and orientation from the content item that is displayed on the display, and where the camera is configured to capture images of an eye of a viewer during such time period when the viewer is viewing the content, in a two dimensional image using visible light. Such system may also include a memory and a processor, where the processor is configured to issue a signal to display the content item on the electronic display during the period of the capture of the images, and is configured to measure a change in a location of the eye between a first image and an image. The processor may be configured to detect a pattern of the changes in location of the eye in the images. In some embodiments, the processor may be configured to compare the detected pattern to a known pattern of eye movements as one or more characteristics may be stored in the memory. In some embodiments, the detected pattern may be correlated with a calculated level of interest of the user in the content being displayed, and the content that is displayed may be associated with a level of interest by such viewer in such content. In some embodiments, the processor may calculate a reliability score or confidence level of the detection of the change in the location of the eye in the various images.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for detecting patterns of eye movements in a series of captured images, the method comprising:
capturing a series of images of at least one eye of a viewer, said series comprising at least a first image frame and a second image frame;
calculating a vector of a movement of said at least one eye between said first frame and said second frame, said vector comprising at least one of a distance of said movement, a direction of said movement, a frequency of said movement, a speed of said movement and a time between said first frame and said second frame;
comparing said vector to at least one characteristic of a plurality of known patterns of eye movements; and
based on the comparison, classifying said movement of said at least one eye as one of said plurality of known patterns of eye movements.

2. The method as in claim 1, wherein an image of said series of images is captured using visible light as a two-dimensional image.

3. The method as in claim 1, wherein an image of said series of images is captured with a portable camera included in a portable housing, said housing including an electronic display suitable for display of a content viewed by said viewer during said capture of said series of said images.

4. The method as in claim 1, wherein said distance is measured in a change in a pixel coordinate of said eye between said first frame and said second frame.

5. The method as in claim 1, wherein said pixel coordinate is measured as a distance of said eye in said first frame from another object on a face of said viewer in said first frame.

6. The method as in claim 1, comprising associating a content viewed by said user during a time of said capturing of said first frame and said second frame, with a known pattern of eye movement and comparing the known pattern of eye movement that is associated with said viewing of said content to said classified one of said plurality of known pattern of eye movements.

7. The method as in claim 6, comprising identifying a pathology associated with said classified first of said plurality of known pattern of eye movements being different from said known pattern of eye movement that is associated with said viewing of said content.

8. The method as in claim 1, comprising determining that said movement of said at least one eye between said first frame and said second frame corresponds to either an actual movement of said eye, or to noise.

9. The method as in claim 8, wherein said determining comprises scoring an accuracy of a detection of said movement between said first frame and said second frame.

10. The method as in claim 1, comprising determining whether said classified movement of said at least one eye differs from said one of said plurality of known pattern of eye movements, and associating said difference with a pathological condition.

11. A system for determining an interest of a user in a displayed content, the system comprising:
an electronic display to display a content item;
an image capture device, said image capture device configured to capture during said time period a plurality of images of an eye of a viewer of said content item in a two dimensional image using visible light,
a memory; and
a processor, said processor to:
issue a signal to display said content item on said electronic display;
measure a change in a location of said eye between a first of said plurality of images and a second of said plurality of images; and
detect a pattern of said changes in location in said plurality of images.

12. The system as in claim 11, wherein said processor is to compare said pattern to a known pattern of eye movements.

13. The system as in claim 11, wherein said processor is to use said detected pattern to calculate an interest of said user in said content item.

14. The system as in claim 13, wherein said memory is to store an association of said user with said content item and said calculated interest.

15. A system for detecting patterns of eye movements in a series of captured images, the system comprising:
a display to display a content item;
a memory; and
a processor, configured to:
calculate a vector of a movement of said at least one eye between said first frame and said second frame, said vector comprising at least one characteristic of a group consisting of a distance of said movement, a direction of said movement, a frequency of said movement, a speed of said movement and a time between said first frame and said second frame;
compare said at least one characteristic of said vector to at least one characteristic of a plurality of patterns of eye movements; and
based on the comparison, classifying said movement of said at least one eye as one of said plurality of patterns of eye movements.

16. The system as in claim 15, further comprising an imager for capturing a series of images of at least one eye, the series comprising at least a first image frame and a second image frame, wherein the imager is a visible light, two-dimensional image camera.

17. The system as in claim 1, wherein the camera is a portable camera and the camera and display are included in a portable housing.

18. The system as in claim 1, wherein the processor is configured to measure the distance in a change in a pixel coordinate of the eye between the first frame and the second frame.

19. The system as in claim 1, wherein the pixel coordinate is measured as a distance of the eye in the first frame from another object on a face of said viewer in said first frame.

20. The system as in claim 1, wherein the processor is further configured to:
associate a content viewed by the user during a time of the capturing of the first frame and the second frame, with a pattern of eye movement, and
compare the pattern of eye movement that is associated with the viewing of the content to the classified one of the plurality of patterns of eye movements.
